(19) United States
(12) Patent Application Publication
(10) Pub. No.: US 2019/0105268 A1
(43) Pub. Date: Apr. 11, 2019

(54) Viscoelastic Gel of Liraglutide Adapted for Once-Weekly or Once Bi-Weekly Administration

(71) Applicant: Sun Pharma Advanced Research Company Limited, Mumbai (IN)

(72) Inventors: Ajay Jaysingh Khopade, Baroda (IN); Arindam Halder, Baroda (IN); Vivek Patel, Baroda (IN)

(73) Assignee: Sun Pharma Advanced Research Company Limited, Mumbai (IN)

(21) Appl. No.: 16/089,776

(22) PCT Filed: Dec. 16, 2016
(86) PCT No.: PCT/IN2016/050447

§ 371 Date: Sep. 28, 2018

(30) Foreign Application Priority Data
Mar. 31, 2016 (IN) 201621011454

(51) Int. Cl. A61K 9/06; A61K 38/26; A61K 47/18; A61K 9/00; A61K 47/24; A61K 47/14; A61K 47/10; A61P 3/10

(52) U.S. Cl. CPC A61K 9/06; A61K 38/26; A61K 47/18; A61K 47/183; A61P 3/10; A61K 47/24; A61K 47/14; A61K 47/10; A61K 9/0019

(57) ABSTRACT

The present invention relates to a viscoelastic gel comprising therapeutically effective amount of liraglutide, wherein the gel does not have a block or a graft copolymer, and wherein the gel is characterized by yield value from 200 Pa to 3000 Pa and flow point from 300 Pa to 3500 Pa. The invention also provides method of controlling blood sugar levels by subcutaneously administering such gel once-weekly or once-biweekly to a subject in need thereof. The method of preparation of such gels are also provided.

### FIELD OF THE INVENTION

The invention relates to viscoelastic gel of liraglutide for once-weekly or once bi-weekly administration, methods of controlling blood glucose levels by administering such viscoelastic gel and use of such viscoelastic gel in the treatment of metabolic diseases. The invention also provides methods of making such viscoelastic gel.

### BACKGROUND OF THE INVENTION

Diabetic mellitus is a disease of metabolic dysregulation, most notably abnormal glucose metabolism, accompanied by characteristic long term complications. It's a chronic disease requiring long term medications. Different parenteral anti-diabetic medications are available in market including human insulin and different GLP-1 agonists.

Liraglutide is approved worldwide, for improvement of glycemic control in patients suffering from Type II diabetes mellitus, as a once daily subcutaneous injection. The molecular formula of liraglutide is C₁₇₂H₂₆₅N₄₃O₅₁. The structure and sequence of liraglutide is shown below—

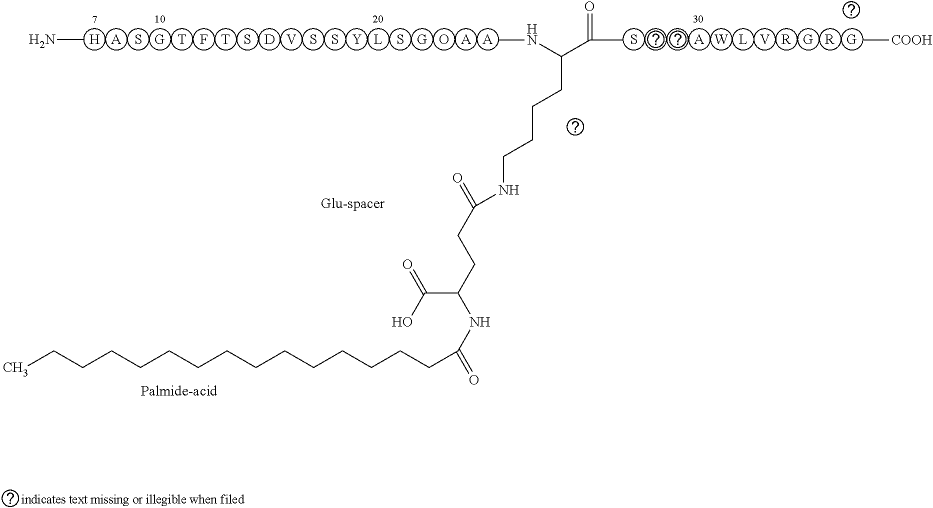

The theoretical molecular mass of liraglutide is 3751.20 Da. It is commercially marketed under the trade name Victoza® which contains 18 mg liraglutide in the form of its anhydrous free-base. Therapy with Victoza® is initiated with a dose of 0.6 mg per day for one week. It may be increased by 0.6 mg to 1.2 mg and if further needed to 1.8 mg once daily. Victoza® is a clear, colorless solution which is supplied as self-injectable, disposable, pre-filled pen that contains a 3 mL solution of liraglutide, equivalent to 18 mg liraglutide, in a glass cartridge. The solution contains following inactive ingredients: disodium phosphate dehydrate, 1.42 mg; propylene glycol, 14 mg; phenol, 5.5 mg; and water for injection. The pH of the solution is adjusted 8.15 with the help of sodium hydroxide and hydrochloric acid. The shelf life is approx. 2 years when stored at 2-8° C.

Liraglutide shows complex solubility behavior. Both liraglutide and its acetate salt are insoluble to slightly soluble in water. It is also slightly soluble in common solvents such as ethanol (1.1. mg/ml) and DMSO. It is soluble in methanol up to 68 mg/ml. The dilute injection of liraglutide is prepared by dissolving liraglutide in water using sodium hydroxide as a base at pH-8.0. Liraglutide remains in a predominantly self-associated heptameric state in concentrations ranging from 0.001-1.2 mM. Without wishing to be bound by theory, the fatty acid side chain on lysine-26 of liraglutide may have a pronounced effect on the interaction strength of the self-associated structure and may be the driving force for the association of the heptameric structure. Peptide self-association and albumin binding at the injection site results in pharmacokinetic profiles suitable for once-daily dosing of a simple, low viscosity formulation in a state-of-the-art needle size (at least as low as 31G). U.S. Pat. No. 6,268,343 disclosed fatty acid acylated GLP-1 agonists, one particular example is liraglutide.

The active ingredient liraglutide is commercially sold in the form of base or its acetate salt. Liraglutide may be prepared by synthetic process or by use of recombinant DNA technology from Saccharomyces Cerevisiae. Crude liraglutide is purified using prep-high performance liquid chromatography with different buffers to give pure fractions. The pure fractions are desalted and lyophilized to obtained fluffy white pure liraglutide.

The liraglutide or acetate of liraglutide slowly dissolves in water whose pH is adjusted to 7-11 using a base. As more liraglutide is added, the powder forms a turbid, non-uniform and incompletely hydrated or gelled mixture which needs further processing to get uniform and transparent gel. The process of dissolving to attain a high concentration of the polypeptide is slow and not suited or ideal for commercial manufacturing.

Long acting composition of GLP-1 agonists are known in the art. An extended release formulation of exenatide, was approved in United States in 2012. This once-weekly formulation consists of exenatide encapsulated in microspheres. US20140220134 disclosed once monthly formulation of exenatide using extended release microspheres suspension comprising poly (lactide-co-glycolide) polymer in a medium chain triglycerides. There is a need to lower the frequency of administration of liraglutide injections in order to increase patient compliance.

### SUMMARY OF THE INVENTION

Present inventors discovered novel lyophilized mixture formed by dissolving liraglutide or its acid addition salt at a first concentration by adding it to a solution of parenterally acceptable amine bases in water for injection, and lyophilizing the aqueous solution so formed. The lyophilized mixture so obtained (liraglutide/base mixture) is useful in preparation of liraglutide aqueous solution at a second concentration without difficulty wherein the second concentration is higher than the first concentration. It is advantageous in that it provides a ready to use quick dissolving liraglutide form for manufacturing concentrated solutions, especially but not limited to the manufacturing of the depot formulations. It exhibits stability during storage, transport and handling. The present inventors have with the use of the novel lyophilized mixture of liraglutide and a parenterally acceptable mixture of liraglutide and a parenterally acceptable amine base further formulated novel viscoelastic gel compositions without the use of synthetically derived block or graft copolymers. The present inventors have also found that with the use of liraglutide in the form of a salt with an inorganic base, a high concentration solution of liraglutide can be made. The solution is sterile if liraglutide salt in sterile form is used and dissolved in sterile water for injection. If the liraglutide salt is not sterile then this aqueous solution can be aseptically filtered, lyophilized and then re-dissolved to get the high concentration liraglutide solution in a sterile form which can be further used for preparing the novel viscoelastic gel of the present invention.

The present invention provides a viscoelastic gel comprising a therapeutically effective amount of liraglutide, wherein the gel does not comprise a block or a graft copolymer or mixtures thereof and wherein the gel is characterized by a yield value from 200 Pa to 3000 Pa and a flow Point from 300 Pa to 3500 Pa. The invention also provides methods of making such viscoelastic gel and use of such viscoelastic gel for treatment of metabolic disorders by once-weekly or once bi-weekly subcutaneous administration. In a preferred embodiment, the viscoelastic gel comprises liraglutide at a concentration from about 10% to 25% by weight of the gel, at least one amphipath and an aqueous vehicle.

In another embodiment, the invention provides a method of controlling the blood glucose levels in a subject in need thereof by subcutaneously administering once-weekly or bi-weekly, a composition in the form of viscoelastic gel comprising a therapeutically effective amount of liraglutide, wherein the gel does not comprise a block or a graft copolymer or mixtures thereof and wherein the gel is characterized by a yield value from 200 Pa to 3000 Pa and a flow Point from 300 Pa to 3500 Pa. The invention also provides use of composition of the invention for treatment of metabolic disease.

### DESCRIPTION OF THE FIGURES

FIG. 1, depicts a graph plotted between the Storage modulus (G′) and Loss modulus (G″) vs shear strain of a representative viscoelastic gel of the invention using an Anton Paar MCR 302 rheometer. The yield value correspond to the limiting value of the linear viscoelastic region and the flow point corresponds to the stress where G′=G″.

FIG. 2. depicts images demonstrating difficulty in preparing an aqueous solution of liraglutide (Example 1b) as compared to the aqueous solution (at second concentration) prepared according to the present invention (Example 3a)

FIG. 3. depicts Cryo-TEM image of viscoelastic gel of Example 4, when analyzed as per Example 11.

FIG. 4 depicts the preclinical efficacy data in db/db mice comparing comparative Example 1a (FIG. 4a) with Gel composition (Gel 10%) of Example 4 (FIG. 4b).

FIG. 5 depicts preclinical efficacy data in db/db mice with weekly subcutaneous administration of Gel composition (Gel 10%) of Example 4 for 28 days, as compared to placebo.

FIGS. 6 and 7 depicts preclinical efficacy data in diet induced diabetic rats with weekly subcutaneous administration of Gel composition (Gel 10%) of Example 4 for 28 days as compared to Victoza®.

FIGS. 8 and 9 depicts preclinical efficacy data in Zucker Diabetic Fatty rats with weekly subcutaneous administration of Gel composition (Gel 15%) of Example 7 for 28 days as compared to Victoza®.

FIG. 10 depicts preclinical efficacy data in db/db mice with weekly subcutaneous administration of Gel composition (Gel 20%) of Example 8 for 28 days.

### DETAILED DESCRIPTION OF THE INVENTION

The present invention provides for a long acting viscoelastic gel of liraglutide, wherein the viscoelastic gel is injected subcutaneously and provides a slow and sustained release of liraglutide over a period of about 6 days to about 10 days, more specifically for about a week, after a single subcutaneous administration. Such long acting viscoelastic gels are advantageous over the daily injections of liraglutide as it provides convenience to patients, thereby increasing patient compliance. The present invention also provides for method of controlling the blood glucose levels over a period of 6 days to about 10 days, more specifically, for about a week, when subcutaneously administered to a subject in need of control of blood glucose levels. Particular embodiments of the present invention are also suitable for once bi-weekly subcutaneous administration.

In one embodiment, the present invention provides a viscoelastic gel comprising a therapeutically effective amount of liraglutide wherein the gel does not comprise a block or a graft copolymer or mixtures thereof, and wherein the gel is characterized by a yield value from 200 Pa to 3000 Pa and a flow Point from 300 Pa to 3500 Pa. The invention also provides methods of making such viscoelastic gel and use of such viscoelastic gel for treatment of metabolic disorders. In a preferred embodiment the gel is administered subcutaneously. In a further preferred embodiment, the gel may be administered once-weekly or once-biweekly.

In a preferred embodiment, the viscoelastic gel comprises liraglutide at a concentration from about 10% to 25% by weight of the gel, at least one amphipath and an aqueous vehicle.

In another embodiment, the invention provides a method of controlling the blood glucose levels in a subject in need thereof by subcutaneously administering, once-weekly or bi-weekly, a viscoelastic gel comprising a therapeutically effective amount of liraglutide, wherein the gel does not comprise a block or a graft copolymer or mixtures thereof and wherein the gel is characterized by a yield value from 200 Pa to 3000 Pa and a flow Point from 300 Pa to 3500 Pa.

The invention also provides use of viscoelastic gel of the invention for treatment of metabolic disease. Each of these embodiments is described in details below.

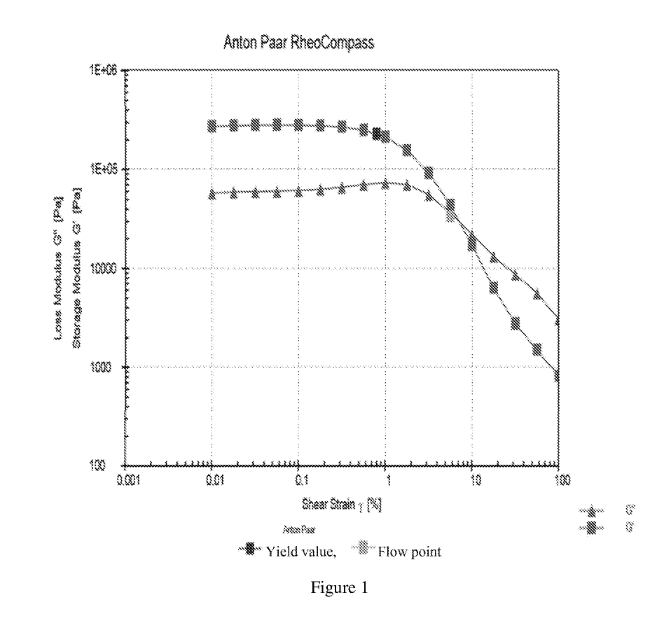

The novel viscoelastic gel of the present invention has high viscosity and behaves like a solid matter without any flow properties on standing, however, the gel is rendered injectable when an external force is applied for example, when forced through a needle by means of a plunger. After injection, the gel regains its consistency at the site of injection, thereby providing a depot effect. The gel may be characterized by rheological parameters using a suitable rheometer performing oscillatory tests. Oscillatory tests involve amplitude sweeps that are performed at different amplitude keeping the frequency and temperature constant. Using these oscillatory tests, the present inventors measured the deformation response and the time-delayed shear stress response and used these measurements to calculate the storage modulus G′ and loss modulus G″. The Storage modulus (G′) and Loss modulus (G″) were then plotted versus shear strain. The typical graph for the viscoelastic gel of one of the representative example of present invention is presented in FIG. 1. The gel was characterized as viscoelastic gel with G′>G″. The present invention provides for viscoelastic gel that provide a similar graph i.e. storage modulus is higher than the loss modulus. Oscillatory tests are also used to determine the yield value and flow point. The yield value correspond to the limiting value of the linear viscoelastic region and the flow point corresponds to the stress where G′=G″. The yield value and the flow point for the representative example of the invention were found to be within the range of 200 Pa to 2500 Pa and from 300 Pa to 3000 Pa respectively. Any suitable rheometer using oscillatory tests may be used to determine the rheology parameters. The present invention provides for viscoelastic gel composition that has a yield value from 200 Pa to 3000 Pa and a flow Point from 300 Pa to 3500 Pa. In a preferred embodiment, the yield value is from 700 Pa to 2000 Pa and a flow point of 1000 Pa to 2500 Pa. In yet another preferred embodiment, the yield value is from 800 Pa to 2500 Pa and a flow point of 1200 Pa to 3000 Pa. The yield value and flow point of the present invention may be measured by any known methods. The present inventors used Anton Paar MCR 302 rheometer using parallel plate fixture with 25 mm diameter at gap of 1 mm, for the measurements. The strain amplitude was varied logarithmically from 0.001 to 100% at constant frequency of 1 Hz (or 10 rad/s) and 25° C. temperature.

The viscoelastic gel comprises therapeutically effective amount of liraglutide. Liraglutide may be present in the gel in the form of base or in the form of its salts or mixtures thereof. Representative examples of salts include salts with suitable inorganic acids or organic acids such as hydrochloric, hydrobromic, formic acid, acetic acid, tartaric acid, ascorbic acid and the like. Representative examples of salts also includes salt with base such as sodium hydroxide, potassium hydroxide, triethanolamine, diethylamine, meglumine, lysine, arginine, alanine, leucine, olamine, tromethamine, choline, taurine, benzamine, methylamine, trimethylamine, propylamine, isopropylamine, and like. In a preferred embodiment, liraglutide may be used in the form of liraglutide acetate. Further the term “liraglutide” also include a mixture of liraglutide base with small amounts of acetic acid for eg. acetic acid may be present in less than 3% of weight of liraglutide and the present invention includes such form of liraglutide. In another preferred embodiment liraglutide may be in the form of its sodium salt. Such forms of liraglutide are commercially available.

The concentration of liraglutide in the viscoelastic gel of the present invention may be from about 5% to 30% by weight of the gel. Preferably, the liraglutide concentration is from 10% to 25% by weight of the gel. The concentration of the liraglutide in the gel determines the volume or the amount of the gel that will be required to administer as a weekly or bi-weekly dose in a subject in need of controlling blood glucose levels. As the weekly or bi-weekly dose of liraglutide is high, a high-concentration liraglutide composition is required so that the injected volume is low. The present inventors have found that the physicochemical property of liraglutide base or its acid addition salt like acetate salt poses problems in making compositions comprising higher concentration of liraglutide. Furthermore, it is to be noted that liraglutide has an auxiliary function in that, it being by itself a polymer of 32 amino acids further derivatized with a lipophilic chain, contributes to the viscous nature of the solution in which it is dissolved. It tends to form highly viscous structure in water at increasing concentrations, thus posing practical difficulty in making a high-concentration liraglutide composition. As the long acting compositions requires a higher dose of liraglutide, difficulty in making high-concentration liraglutide compositions present a challenge. The present inventors have found advantageous methods to increase the loading of liraglutide in a composition thus leading to high concentration compositions. Such high concentration liraglutide compositions are not known in the art. In one of the methods, a lyophilized mixture of liraglutide with a parenterally acceptable amine base is prepared. In another method, a salt of liraglutide with an inorganic base may be used. In either of the methods, either the drug may be used in sterile form or a first aqueous solution of the drug may be prepared and sterilized for use in further steps of preparing the viscoelastic gel. Furthermore, the present inventors were successful in making a high-concentration viscoelastic gel, specifically with 5 to 30% liraglutide, with a yield value within the range of 200 Pa to 3000 Pa and a flow point in the range of 300 Pa to 3500 Pa, that provides a sustained release of liraglutide over a period of time thus providing for long action for about 6 days to about 10 days after a weekly subcutaneously administration. The present invention can also provide viscoelastic gel suitable for bi-weekly i.e. once every 15 days subcutaneous administration.

The present inventors have found that a block or graft copolymer or mixtures thereof such as required in the compositions disclosed in the co-pending application PCT/IN2016/050185 are not essential in the viscoelastic gel according to the present invention. The compositions claimed in PCT/IN2016/050185 were found to be highly viscous with semi solid consistency and were rendered injectable when forced through a needle. The present invention however does not require a block or a graft copolymer or mixtures thereof. This is advantageous because diabetes therapy is a chronic therapy and minimum number of excepients is thus desirable. The present invention avoids the use of synthetically derived copolymers.

In a preferred embodiment, the viscoelastic gel comprises therapeutically effective amount of liraglutide at a concentration of 10-25%, at least one amphipath, and an aqueous vehicle and does not comprise a block or graft copolymer or mixtures thereof. In a further preferred embodiment, the composition may further comprise a parenterally acceptable amine base.

The term “amphipaths” that may be used in the composition of the present invention refers to compounds which contain both a hydrophilic or hydrophilic (lipophilic) group. The amphipaths suitable for use in the viscoelastic gel of the present invention include but not limited to mono and diglycerides, polyglycerized fatty acids, polyethoxylated fatty acids, PEG-fatty acid mono and di-esters and mixtures thereof, PEG glycerol fatty acid esters, Alcohol-oil transesterification products, propylene glycol fatty acid esters, mixtures of propylene glycol esters and glycerol esters, PEG sorbitan fatty acid esters, PEG alkyl ethers, PEG alkyl phenols, and sorbitan fatty acid esters. Examples of mono di and triglycerides for use as amphipath in the viscoelastic gel of present invention are glycerol monoleate, glycerol monolaurate, glycerol monopalmitate, glycerol monostearate, glycerol acetate, glycerol laurate, glycerol caprylate, glycerol caprate, glyceryl monostearate, glycerol dioleate, caprylic acid mono, diglycerides, dicaprin, dimyristin, dipalmitin, glyceryl dilaurate, glycerol trioleate, glycerol tristearate and glycerol esters of fatty acids. Examples of polyglycerized fatty acids for use as amphipath in the viscoelastic gel of present invention are polyglyceryl-2, 4, 10, stearate, polyglyceryl-2, 3, 4, 6, 10 oleate, polyglyceryl-2 isostearate, polyglyceryl-10 laurate, polyglyceryl-6 ricinoleate, polyglyceryl-10 linoleate, polyglyceryl-2, 3 dioleate, polyglyceryl-3 distearate and polyglyceryl-10 trioleate. Examples of polyethoxylated fatty acids for use as amphipath in the viscoelastic gel of present invention are PEG 1-10 Stearate, PEG 2oleate, PEG 4 laurate, PEG 4-100 monooleate, PEG 4-monostearate, Examples of PEG-fatty acid di-esters and mixtures with mono-esters for use as amphipath in the viscoelastic gel of present invention are diesters of lauric acid, oleic acid, stearic acid, palmitic acid with different grades of PEG such as PEG-4 dilaurate, PEG-4-dioleate, PEG-4 distearate, PEG-10 dipalmitate, PEG-6 dilaurate, PEG-6 dioleate, PEG-6 distearate, PEG-8 dilaurate, PEG-8 dioleate, PEG-8 distearate, PEG-12 dilaurate, PEG-12 dioleate, PEG-12 distearate, PEG-32 dilaurate, PEG-32 dioleate, PEG-32 distearate. Examples of PEG glycerol fatty acid esters for use as amphipath in the viscoelastic gel of present invention are esters of lauric acid, oleic acid, stearic acid with different grades of PEG such as PEG-15 glyceryl laurate, PEG-20 glyceryl laurate, PEG-20 glyceryl stearate, PEG-20 glyceryl oleate and PEG-15 glyceryl oleate. Examples of Alcohol-oil transesterification products for use as amphipath in the viscoelastic gel of present invention are PEG-5-10 castor oil, PEG-5, 7, 10 hydrogenated castor oil, PEG-6 peanut oil, PEG-6 kernel oil, PEG-6 corn oil, PEG-20 corn glycerides, PEG-8 and 6 caprylic/capric glycerides, Pentaerythrityl tetraisostearate Pentaerythrityl distearate, Pentaerythrityl tetraoleate, Pentaerythrityl tetrastearate Pentaerythrityl tetracaprylate/tetracaprate. Examples of propylene glycol fatty acid esters for use as amphipath in the viscoelastic gel of present invention are propylene glycol monocaprylate, propylene glycol monolaurate, propylene glycol oleate, propylene glycol myristate, propylene glycol ricinoleate, propylene glycol dicaprylate/dicaprate, propylene glycol dioctanoate, propylene glycol dilaurate, propylene glycol distearate and propylene glycol dicaprylate. Examples of mixtures of propylene glycol esters and glycerol esters for use as amphipath in the viscoelastic gel of present invention are esters of oleic and stearic acid. Examples of PEG sorbitan fatty acid esters for use as amphipath in the viscoelastic gel of present invention are PEG-4, 6, 10 sorbitan monolaurate, PEG-10 sorbitan monopalmitate, PEG-4, 6, 8, 10 sorbitan monostearate, PEG-5, 6, 10 sorbitan monooleate, PEG-6 sorbitan tetraoleate, PEG-6 sorbitan tetrastearate, PEG sorbitan hexaoleate, PEG sorbitan hexastearate. Examples of PEG alkyl ethers for use as amphipath in the viscoelastic gel of present invention are PEG oleyl ethers, PEG lauryl ethers, PEG cetyl ethers and PEG stearyl ethers. Examples of PEG alkyl phenols for use as amphipath in the viscoelastic gel of present invention are PEG-10 nonyl phenol and PEG-15 octylphenol ether. Examples of sorbitan fatty acid esters for use as amphipath in the viscoelastic gel of present invention are sorbitan monopalmitate, sorbitan monooleate, sorbitan monostearate, sorbitan monolaurate, sorbitan trioleate, sorbitan tristearate, sorbitan sesquistearate and sorbitan sesquioleate.

Amphipaths suitable for use in the viscoelastic gel may also include a phospholipid. Phospholipids used in present invention may be obtained from plant source, animal sources or synthetic source. Natural phospholipids can be obtained from vegetable sources like, e.g., soybeans, rape (canola) seed, wheat germ, animal material, like egg yolk, milk etc. Examples of natural phospholipids are soya lecithin, egg lecithin, enzyme-modified natural phospholipids such as monoacyl-phosphatidylcholine (lyso PC), soy PE, soy PG, egg PG, and saturated analogs. Examples of synthetic phospholipid are PEG-ylated phospholipids and the cationic phospholipid 1,2-diacyl-P—O-Ethylphosphatidylcholine or mixtures thereof. Examples of semisynthetic phospholipids include Dipalmitoylphosphatidylcholine (DPPC), 1-palmitoyl-2-oleoyl phosphatidylcholine (POPC), dioleoyl phosphatidylcholine (DOPC), dilinoleoyl phosphatidylcholine (DLiPC), lysophosphatidylcholine (LPC), 1-palmityol-LPC (PaLAC), 1-oleoyl-LPC (OiLPC), Phosphatidylethanolomine (PE), plasmenyl ethanolamine (PlaE), glycerol acetal of plasmenyl ethanolamine (GAPlaE), didodecyl phosphatidyl ethanolamine (DDPE), dielaidoyl phosphatidyl ethanolamine (DEPE), dioleoyl phosphatidyl ethanolamine (DOPE), dilinoleoyl phosphatidyl ethanolamine (DLiPE), dioleoyl phosphatidyl-N-monomethylethanolamine (DOPE-Me), dophosphatidylglycerol (DPG), Phosphatidylglycerol (PG), Phosphatidylserine (PS), Phosphatidylinositol (PI), Preferred phospholipid includes phosphatidyl choline. Preferably phosphatidyl choline is soy phosphatidyl choline (SPC) or mixtures thereof. Amphipaths suitable for use in the viscoelastic gel may include nonionic and zwitterionic surfactants, monoglyceride and sphingolipids and phospholipids as described in Fontell et al., Colloidal & Polymer science, 268: 264-285 (1990)

Preferably, the amphipath suitable for use in the composition of the present invention may be selected from glyceryl monooleate, glyceryl dioleate, glyceryl trioleate, plyglyderyl-3-dioleate, and phosphatidylcholine and mixtures thereof. In a further preferred embodiment, the amphipath is a mixture of glyceryl monooleate, glyceryl dioleate, glyceryl trioleate and phosphatidylcholine. The mixture may include small amount of fatty acid, preferably oleic acid. For instance, Pharmacopoeial grade, commercially available amphipath available by the trade names IMWITOR® may be used. IMWITOR® 948 is manufactured by esterification of plant derived glycerol with vegetable sourced fatty acids, mainly oleic acid, which contains 40% of nominal content of monoglyceride in ratio of monoglyceride (32.0-52.0%), diglyceride (30.0-50.0%) and triglyceride (5.0-20.0%). The mixture of mono, di and triglyceride are available in different ratios as per the below nominal content of monoglyceride and available as different grades of IMWITOR®. IMWITOR® has also been referred to as Glyceryl oleates in specific examples.

| | IMWITOR ® | | |
|---|---|---|---|
| | Nominal content of monoglyceride (%) | | |
| | With about 40% | With about 60% | With about 90% |
| Monoglyceride | 32.0-52.0 | 55.0-65.0 | 90.0-101.0 |
| Diglyceride | 30.0-50.0 | 15.0-35.0 | <10.0 |
| Triglyceride | 5.0-20.0 | 2.0-10.0 | <2.0 |

Generally, the weight ratio between a phospholipid and a mixture of a mono, di and triglycerides thereof in the present viscoelastic gel is 50:50. The amphipath is present in the viscoelastic gel of the invention at a concentration from 40% to 60% by weight of the gel. Preferably, amphipath is present in the viscoelastic gel of the invention at a concentration from at a concentration from 45% to 55% by weight of the gel.

The aqueous vehicle that may be used in the viscoelastic gel of the present invention includes a mixture of water and a water miscible solvent. Water is used for dissolving water soluble or water miscible components, and at least one water miscible solvent may be used for dissolving amphipaths, particularly amphipaths that are not water soluble. An aqueous vehicle suitable for use in the viscoelastic gel include but not limited to water, alcohols, ethers, esters and ketones or mixtures thereof. Alcohols may include class of vehicles and include monols, diols and polyols, for eg. ethanol, glycerol, polyethylenglycol or propylene glycol. Suitable ethers may include diethyl ether, glycofurol, diethylene glycol and polyethylene glycol. Unlike conventional liquid and semi-solid compositions, the viscoelastic gel of the present has a lower concentration of the liquid vehicle than the total concentration of other components of the invention. The aqueous vehicle may be present at a concentration of 20% to 40%. In a preferred embodiment, the aqueous vehicle may be present in a concentration of 30% to 35%. Preferably, the aqueous vehicle is selected from water, ethanol, propylene glycol, glycofurol, glycerol and mixtures thereof.

In a preferred embodiment, the gel composition may comprise a parenterally acceptable amine base. The parenterally acceptable amine base is especially required in compositions using liraglutide base or an acid addition salt of liraglutide like an acetate salt. As stated before, liraglutide base or acetate salt present problems with respect to solubility of the drug in water. Thus, for preparing a viscoelastic gel using liraglutide base or an acid addition salt of liraglutide, a parenterally acceptable amine base can be used in accordance with the present invention so as to prepare a high concentration solution of liraglutide. The parenterally acceptable amine base may be present in the viscoelastic gel of the invention as a lyophilized mixture with liraglutide. The invention also provides an advantageous lyophilized mixture of liraglutide and a pharmaceutically acceptable amine base. Inventors have surprisingly found that a viscoelastic gel comprising liraglutide at a concentration from 10% to 25% can be prepared when liraglutide is present as a lyophilized mixture with a parenterally acceptable amine base in the composition.

The parenterally acceptable amine base is selected from triethanolamine, diethylamine, meglumine, ornithine, lysine, arginine, alanine, leucine, diethylethanolamine, olamine, triethylamine, tromethamine, glucosamine, choline, trimethyl maine, taurine, benzamine, trimethyl ammonium hydroxide, epolamine methylamine, diemthylamine, trimethylamine, methylethanolamine, propylamine, isopropylamine, and like. Preferably, the parenterally acceptable amine base is selected from tromethamine, arginine, histidine, lysine, guanidine, epolamine, glucosamine and meglumine. More preferably, the parenterally acceptable amine base is selected from tromethamine and arginine.

In another embodiment, the invention provides a method of controlling the blood glucose levels in a subject in need thereof, by subcutaneously administering, in once-weekly or bi-weekly, a composition in the form of viscoelastic gel comprising a therapeutically effective amount of liraglutide, wherein the gel does not comprise a block or a graft copolymer or mixtures thereof and wherein the gel is characterized by a yield value from 200 Pa to 3000 Pa and a flow Point from 300 Pa to 3500 Pa.

The term “controlling the blood glucose level in a subject” refers to reducing the concentration of blood glucose in a subject in need thereof, towards a normal physiological range, and thus provide efficacy in the treatment of diabetes or related disorders. The normal physiological range of glucose is well known to those skilled in the art. The term “subject in need thereof” refers to the subjects who require normalization of blood glucose levels to physiological levels, particularly subjects requiring treatment of diabetes or related disorders. The term “subject” used herein refers to a mammalian, including human and animals.

In a preferred embodiment, the method of the present invention provides subcutaneous administration of gel composition comprising about 5% to about 30% of liraglutide. In a preferred embodiment, the concentration of liraglutide is from about 10% to 25%.

The present inventors have found a method to control blood glucose level using a viscoelastic gel that regains its consistency upon injection at the injection site and provides a depot, from which liraglutide is released slowly into plasma for eliciting the glucose lowering effect over a period of about 6 days to 10 days, preferably for about a week. The method of the present invention may also be used to provide a glucose lowering effect for about 15 days after bi-weekly administration.

In a further preferred embodiment, the method of the present invention provides subcutaneous administration of gel composition comprising liraglutide at a concentration from about 10% to about 25% of liraglutide, at least one amphipath, and an aqueous vehicle. The term amphipath is as defined above. The amphipath may present at a concentration from 40% to 60% by weight of the gel. In a preferred embodiment, the amphipath may be used at a concentration from 45% to 55% by weight of the gel. The specific examples of amphipath that may be used in the method of the present invention may be selected from the examples of amphipath provided above. Preferably, the amphipath is selected from glyceryl monooleate, glyceryl dioleate, glyceryl trioleate, polyglyceryl-3-dioleate, phosphatidylcholine and mixtures thereof. More Preferably, the amphipath is a mixture of glyceryl monooleate, glyceryl dioleate, glyceryl trioleate and phosphatidylcholine. The aqueous vehicle that may be present at a concentration from 20% to 40% by weight of the gel. In a preferred embodiment, the aqueous vehicle may be present in a concentration of 30% to 35%. The aqueous vehicle may include a mixture of water and a water miscible solvent. Water is used for dissolving water soluble or water miscible components, and at least one water miscible solvent may be used for dissolving amphipaths, particularly amphipaths that are not water soluble. Preferably, the aqueous vehicle is selected from water, ethanol, propylene glycol, glycofurol and mixtures thereof.

The viscoelastic gel of the invention may be administered by subcutaneous or intramuscular injection. More preferably, the viscoelastic gel of the invention may be administered by subcutaneous injection.

The present invention also provides use of viscoelastic gel of the present invention in treatment of metabolic disorders. More specifically, the composition may be useful in disease which benefit from a control in glucose levels for e.g. hyperglycemia, type II diabetes, hypertriglyceridemia, hypercholesterolemia, cardiovascular disorders, obesity, renal disorders, CNS disorders, ocular disorders etc.

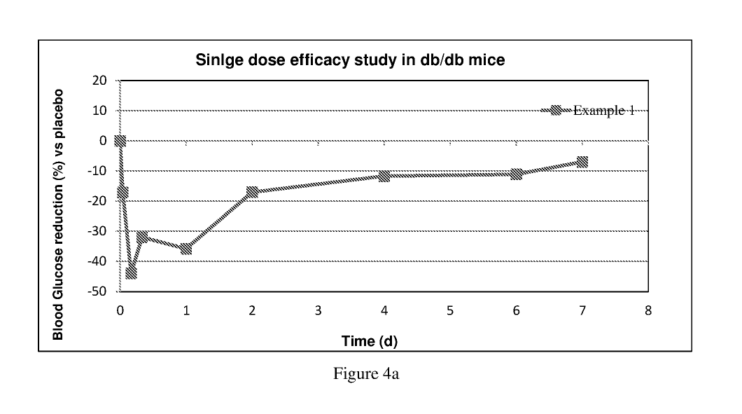

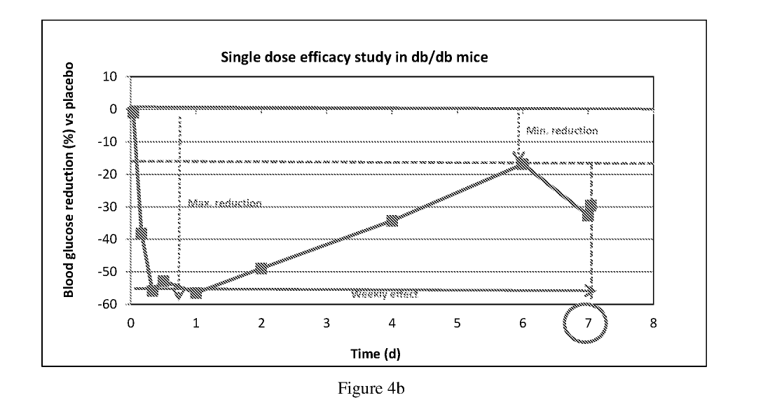

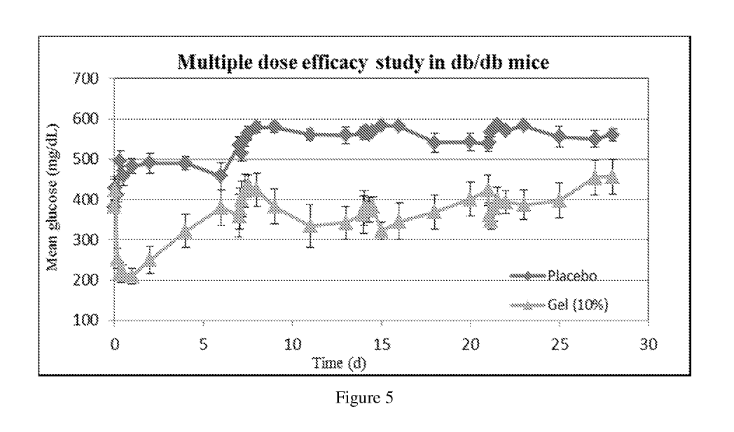

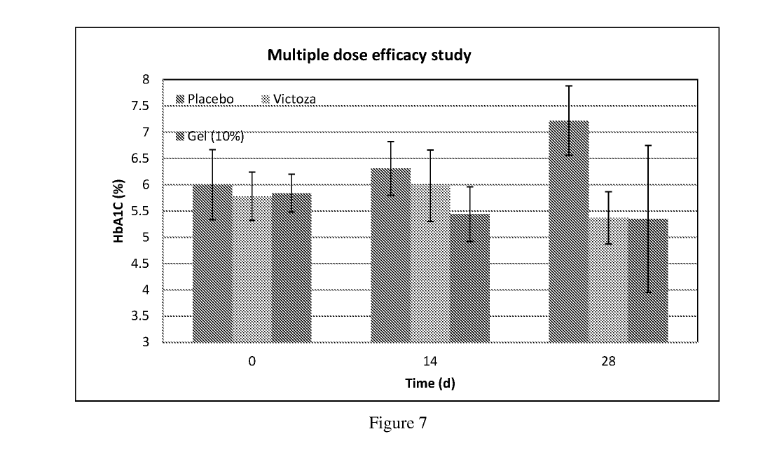

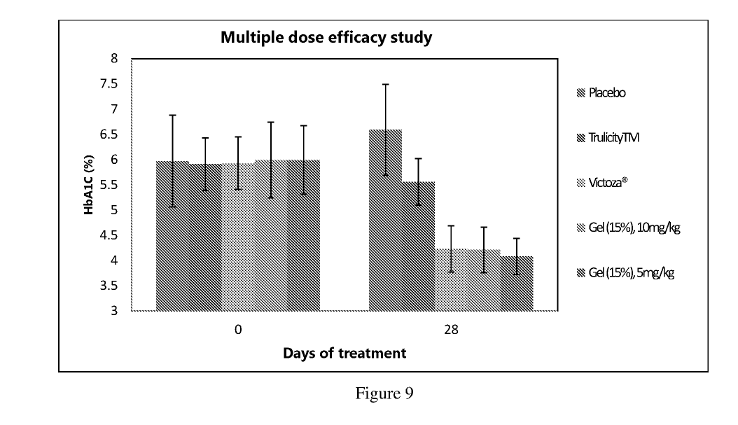

The present inventors have tested the efficacy of the representative viscoelastic gel composition of the present invention in preclinical studies and the data is provided in examples 12-15 below. The viscoelastic gel composition was found to provide lowering of blood glucose levels upon single subcutaneous administration to diabetic mice, for about a week (FIG. 4). Further, a steady decrease in blood glucose levels was found even in multiple dose study (See FIGS. 5, 6, 8 and 10). Further, percentage change in blood glucose on administration of viscoelastic gel of present invention was found to be comparable or better than daily administration of Victoza® or weekly administration of Trulicity™. The viscoelastic gel of the invention are also effective in reducing Hb1Ac levels. (FIGS. 7 and 9). Thus, the viscoelastic gel of the present invention are useful for prevention or treatment of type 2 diabetes, hyperglycemia or impaired glucose tolerance as well as for treatment of metabolic diseases like obesity.

The viscoelastic gel of the present invention may be prepared by methods known in the art. The process used by the present inventors to prepare the composition of the present invention is presented herein below. The process comprises broadly of two steps: First step being preparation of lyophilized mixture of liraglutide with an amine base and second step involves preparing an aqueous solution using the lyophilized mixture of first step and further mixing it with the non-aqueous solution of an amphipath. For compositions wherein liraglutide is used as a base or an acid addition salt of liraglutide like an acetate salt, the first and the second step may be performed as stated hereinbelow. It is possible to make high concentrations of liraglutide from 5% to 30% directly without the problems associated with liraglutide acetate if instead a liraglutide salt with an inorganic base is used. Sodium salt of liraglutide has recently become available commercially in sterile form. The same can be dissolved in water for injection to directly yield the high concentration aqueous solution in a sterile form. If the liraglutide salt is not sterile then this aqueous solution can be aseptically filtered, lyophilized and then re-dissolved to get the high concentration liragluide solution in a sterile form The process is described in stepwise manner herein below.

The lyophilized mixture of the present invention can be prepared by a process comprising:

a. preparing a solution of parenterally acceptable amine base in water for injection.

b. adding liraglutide or its acid addition salt to solution of step (a) while stiffing the solution to form the first aqueous solution at first concentration.

c. Optionally sterilizing the first aqueous solution of step (b) by aseptic filtration

d. Lyophilizing the solution to obtain a lyophilized mixture.

wherein the amount of parenterally acceptable amine base is such that the pH of the first aqueous solution is in the range from about 6.7 to about 10, and wherein the lyophilized mixture is adapted for preparing an aqueous solution at a second concentration wherein the second concentration is higher than the first concentration.

Steps a and b of the process for preparation of a lyophilized mixture are carried out using the conventional techniques which involves dissolving and mixing the ingredients as appropriate to give the desired end product. A required amount of water for injection is taken in a suitable vessel. Weighed amount of parenterally acceptable amine base is dissolved in water for injection under gentle stirring. Liraglutide is added slowly and stirred to disperse. Parenterally acceptable amine base (as solid or solution) is further added while stiffing until a pH of the aqueous solution of first concentration is in the range from about 6.7 to about 10 is obtained and the solution becomes clear. Alternatively the quantity of parenterally acceptable amine base can be optimized and then to the solution of the parenterally acceptable amine base in water for injection liraglutide may be gradually added with stiffing to yield a solution having is in the range from about 6.7 to about 10. Preferably, the pH of the solution is in the range from about 6.7 to about 8.5, more preferably in the range from about 7.0 to about 8.3. The amine base is typically used from 1:1 to 1:6 molar ratio to liraglutide, For example, tromethamine is used from 3% to 18%, arginine or histidine are used from 4.0% to 25%. Additional amine may be required if the acid addition salt of liraglutide is used in the composition. Optionally the first aqueous solution may be sterilized by aseptic filtration, preferably by filtering through a 0.2μ membrane filter.

The “first concentration” herein refers to the concentration of the liraglutide aqueous solution prepared by dissolving liraglutide or its acid addition salt in an aqueous vehicle containing parenterally acceptable amine base for the preparation of lyophilized mixture of liraglutide. The first concentration is limited by its ability to be able to be filtered through 0.2 μm filter. Typically, the first concentration may be in the range from about 0.5 to 30% w/w, preferably in the range from about 5 to 20% w/w. The ability to make a high concentration also depends on the type of base used.

The first aqueous solution of first concentration is filled in containers such as individual vials to the desired volume and the vials are subjected to a lyophilization process. For the bulk manufacturing, the solution is filled in freeze-drying trays/containers to the desired volume and are subjected to a lyophilization process. Freeze-drying trays such as Gore® Lyogaurd® trays (mfg by W.L. Gore & Associates Inc.), which are fully enclosed, single-use disposable containers that use a unique expanded polytetrafluoroethylene (ePTFE) membrane technology to both prevent cross-contamination and fly-out, and enable the free exchange of moisture vapor during lyophilization are used. The lyophilization process is carried out in a conventional manner Trays containing lyophilized drug are individually packed in pre-sterilized paper bags/aluminum pouches, sealed and stored at −20° C.

The inventors have discovered that lyophilized mixture of the invention is a ready-to-use quick dissolving liraglutide form which can be readily used for the manufacturing of concentrated solutions, especially but not limited to the manufacturing of a depot composition. Thus, a long acting composition, comprising liraglutide at a high concentration, for example from 10% to 25% can be easily prepared and the process of its preparation is ideal for commercial manufacturing. The present invention thus provides lyophilized mixture of liraglutide and a parenterally acceptable amine base, in particular preference a lyophilized mixture of liraglutide with tromethamine and a lyophilized mixture of liraglutide with arginine.

In another aspect, the gel composition of present invention comprising lyophilized mixture of liraglutide and parenterally acceptable amine base comprises:

a) preparing a lyophilized mixture by the process of the invention as discussed above.

b) preparing a aqueous solution at a second concentration by dissolving the lyophilized mixture in water for injection, and if necessary sterilizing the solution by aseptic filtration.

c) preparing a non-aqueous solution of amphipath in a water-miscible solvent, and sterilizing the solution by aseptic filtration.

d) aseptically adding the solution of step (c) to the solution of step (b) and mixing.

The “second concentration” herein refers to concentration of liraglutide aqueous solution prepared by dissolving the lyophilized mixture of the invention comprising liraglutide and a parenterally acceptable amine base. The second concentration may be prepared in the range from about 1 to 60% w/w preferably, in the range from about 15 to 50% w/w.

Step b) for the making of a gel composition of the invention adapted for once weekly or biweekly administration involves preparing a aqueous solution at a second concentration by dissolving the lyophilized mixture as prepared by the process of the invention in water for injection. The solution is prepared using the conventional techniques of the pharmaceutical industry which involves dissolving and mixing the ingredients as appropriate to give the desired end product. This aqueous solution at a second concentration may be in the form of solution, or semi-viscous solution or clear gel depending on the concentration of liraglutide. Optionally the aqueous solution at a second concentration may be sterilized by aseptic filtration, preferably by filtering through a 0.2μ membrane filter.

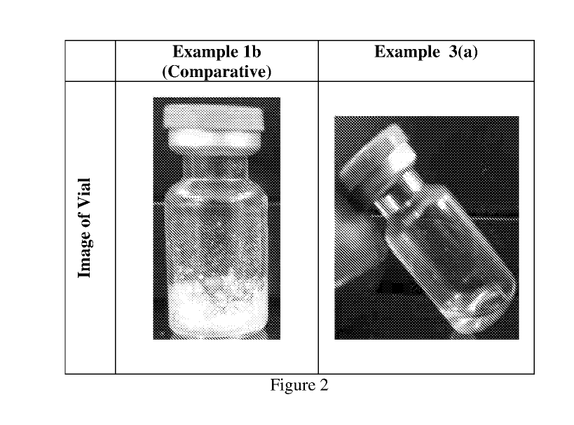

The lyophilized mixture of the invention provides advantageous properties in the preparation of viscoelastic gel of liraglutide adapted for once weekly or bi-weekly administration as compared to liraglutide base or its acid addition salt. For example, it is ready to use powder that allows formulating any desired concentration by a single step process. The lyophilized mixture of the invention exhibits enhanced solubility and improved stability under ambient storage, transport and handling. Depending on the type of base, higher gelling concentrations of the solutions are achieved. This is important aspect of the current invention as it is preferred for formulating a gel composition adapted for once weekly or bi-weekly administration with reduced injection volume. An aqueous solution prepared using this lyophilized mixture is the aqueous solution at second concentration and is clear transparent and stable at high concentration of liraglutide when kept at a temperature of 2-8° C. This is evident from FIG. 2 wherein solution made using the lyophilized mixture of the invention remains clear. Whereas when an aqueous solution of liraglutide is prepared by adding liraglutide acetate in a solution of water and tromethamine, as in example 1(b), resulted in a turbid, non-uniform and incompletely hydrated or partially gelled mixture, whereas an aqueous solution prepared using lyophilized mixture forms clear gel. Another aspect of this invention is the choice of parenterally acceptable amine base allows making higher concentration of solution or gelling. For example, Liraglutide with arginine remains flowable liquid at 28% concentration and forms a gel at 39% concentration while, liraglutide with tromethamine forms gel at 28% concentration.

Step c) of the process for making a gel composition of the invention for once weekly or bi-weekly administration involves preparation of solution of amphipaths in a water-miscible solvent. The amphipath in the viscoelastic gel is present at a concentration from 40% to 60% by weight of the gel. This is prepared by dissolving the amphipaths in water-miscible solvent at a temperature of 60-70° C. under stiffing. The process involves conventional method of mixing by using stirrer. Typically, required amounts of solvents are taken in a tank fitted with a stirrer. Lipids are slowly added with stiffing while maintaining the temperature of the mixture at 60-70° C. Optionally, this non-aqueous solution of amphipaths may be sterilized by aseptic filtration, preferably by filtering through a 0.2μ membrane filter.

Step d) of the process involves adding the non-aqueous solution of amphipaths to the aqueous solution at second concentration using stiffing to form a gel composition in the form of a viscoelastic gel.

### EXAMPLES

The compositions of the present invention example are described in detail. However, it is to be noted that the present disclosure is not limited to the illustrative examples but can be realized in various other ways.

### Example 1a (Comparative)

| | | | |
|---|---|---|---|
| | | Quantity | % |
| Sr. No. | Ingredients | (mg) | (w/w) |

| | |
|---|---|
| A | Phase I |

| | | | |
|---|---|---|---|
| 1 | Liraglutide | 5 | 3.91 |
| 2 | Tromethamine | 0.5 | 0.39 |
| 3 | Polysorbate-80 | 2.5 | 1.5 |
| 4 | Water for Injection | 20 | 15.6 |
| | Total Phase I | 28 | |

| | |
|---|---|
| B | Phase II |

| | | | |
|---|---|---|---|
| 1 | Soy Phosphatidyl choline (SPC) | 42.5 | 33.2 |
| 2 | Glycerol Oleates in ratio: - | 42.5 | 33.2 |
| | GMO:GDO:GTO = 44:42:9 | | |
| 3 | Ethanol | 10 | 7.81 |
| 4 | Propylene Glycol | 5 | 3.9 |
| | Total Phase II | 100 | — |

| | |
|---|---|
| | Phase III: Concentrated gel phase |
| C | (Mixture of Phase I and Phase II): |

| | | | |
|---|---|---|---|
| 1 | Phase I | 28 | — |
| 2 | Phase II | 100 | — |
| | Total concentrated gel phase | 128 | — |

Manufacturing process of composition:
(A) Phase I: Liraglutide (5 mg), tromethamine (0.5 mg) and polysorbate-80 (2.5 mg) was dissolved in water for injection (20 mg) and kept at 20-25° C.
(B) Phase II: Soy Phosphatidyl choline (SPC) (42.5 mg), Glycerol Oleate (mixture of mono, di and tri oleate and free fatty acid) (Glyceryl monooleate (GMO):Glyceryl diooleate (GDO):Glyceryl trioleate (GTO) = 44:42:9) (42.5 mg), Propylene Glycol (5 mg), Ethanol (10 mg) ware mixed and dissolved at 60-70° C. temperature under stirring for15-20 min.
(C) Phase III: Phase II was added to phase I using stirring at 60-70° C. temperature to form concentrated gel phase. The yield value and flow point was measured by Anton Paar MCR 302 rheometer using parallel plate fixture with 25 mm diameter at gap of 1 mm. The strain amplitude was varied logarithmically from 0.001 to 100% at constant frequency of 1 Hz (or 10 rad/s) and 25° C. temperature. The Yield value was determined to be 46.95 Pa and Flow point to be 78.74 Pa. The present example was tested in preclinical studies according to example 12, wherein the gel product was administered subcutaneously to diabetic mice. As shown in FIG. 4a, the gel was not effective in lowering blood glucose levels as compared to example of the invention (FIG. 4b).

### Example 1b (Comparative)

Preparation of aqueous solution of liraglutide acetate

| | | Quantity | Quantity |
|---|---|---|---|
| S. No. | Ingredients | (mg) | (% w/w) |
| 1 | Liraglutide (acetate) | 48 | 27.58 |
| | equivalent to liraglutide | | |
| 2 | Tromethamine | 6 | 3.44 |
| 3 | Water for Injection | 120 | 68.96 |

Tromethamine was dissolved in water for injection in a vial under gentle stirring. When liraglutide acetate when added to above solution, it resulted in a turbid, non-uniform and incompletely hydrated or partially gelled mixture as shown in FIG. 2

### Example 2

Preparation of aqueous solution at second concentration
with liraglutide and tromethamine and lyophilizing
the same to make a lyophilized mixture

| S. No. | Ingredients | Quantity (% w/w) |
|---|---|---|
| 1 | Liraglutide (acetate) | 2.5 |
| | equivalent to liraglutide | |
| 2 | Tromethamine | q.s to pH 6.5-8.5 |
| 3 | Water for Injection | 100 |

Water for injection was taken in a vial. An amount of tromethamine was dissolved in water for injection under gentle stiffing. Liraglutide acetate was added to above solution with stiffing. The pH was measured and tromethamine was added, if required, while stiffing until a pH of 6.5-8.5 was obtained and the solution was clear. The aqueous solution of first concentration (2.5% w/w) of liraglutide was then filtered with 0.2 μm membrane filter, filled into vials and lyophilized. The assay for liraglutide in the lyophilized mixture was 91.1%.

### Example 3

### Preparation of Aqueous Solution at Second Concentration from Lyophilized Mixture Containing Liraglutide with Tromethamine

Three different solutions at second concentration were prepared as follows:

a) Aqueous solution at of liraglutide
at second concentration (11.1% w/w)

| | Ingredients | Quantity |
|---|---|---|
| S. No. | Aqueous solution at second concentration | (mg) |
| 1 | Lyophililized mixture of Example 2 | 15* |
| 2 | Sterile Water for Injection | 120 |

*equivalent to liraglutide base

The Lyophilized mixture of Example 2 was dissolved in sterile water for injection resulting in aqueous solution with second concentration (containing liraglutide at a concentration of 11.1% w/w) which was clear and transparent as shown in FIG. 2. This was kept at 20-25° C.

(b) Aqueous solution at of liraglutide
at second concentration (20% w/w)

| | Ingredients | Quantity |
|---|---|---|
| S. No. | Aqueous solution at second concentration | (mg) |
| 1 | Lyophililized mixture of Example 2 | 30* |
| 2 | Sterile Water for Injection | 120 |

*equivalent to liraglutide base

The Lyophilized mixture of Example 2 was dissolved in sterile water for injection resulting in aqueous solution with second concentration (containing liraglutide at a concentration of 20% w/w). This was kept at 20-25° C.

(c) Aqueous solution at of liraglutide
at second concentration (28.6% w/w)

| | Ingredients | Quantity |
|---|---|---|
| S. No. | Aqueous solution at second concentration | (mg) |
| 1 | Lyophililized mixture of Example 2 | 48* |
| 2 | Sterile Water for Injection | 120 |

*equivalent to liraglutide base

Lyophilized mixture of Example 2 was dissolved in sterile water for injection resulting in aqueous solution with second concentration (28.6% w/w). This was kept at 20-25° C.

### Example 4

Preparation Gel composition using aqueous
solution of second concentration

| | | Quantity |
|---|---|---|
| S. No. | Ingredients | % w/w |
| 1 | Liraglutide | 10 |
| | Non-aqueous solution of amphipaths | |
| 1 | Soy Phosphatidylcholine (Lipoid S 100) | 26.9 |
| 2 | Glycerol Oleates in ratio: - GMO:GDO:GTO = | 26.9 |
| | 44:42:9 (IMWITOR ® 948) | |
| 3 | Ethanol Absolute 99.9% (Commercial Alcohols) | 6.3 |
| 4 | Propylene Glycol USP | 3.2 |
| | Total preparation | 100 |

Non-aqueous solution of amphipaths - Soy Phosphatidylcholine (Lipoid S 100), Glycerol Oleate (IMWITOR ® 948, mixture of mono, di and tri oleate and free fatty acid) were dissolved in Propylene Glycol and Ethanol at 60-70° C. with stirring for 15-20 min and filtered with 0.2 μm membrane filter.
Gel composition- Non-aqueous solution of amphipaths was added to 36.7% w/w of aqueous solution at second concentration of Example 3(c) (comprising liraglutide at a concentration of 28.6%) and mixed using stirrer, to form gel using stirring at 50-70° C. The lipid gel is yellowish viscous of semisolid consistency. The yield value and flow point were measured by Anton Paar MCR 302 rheometer using parallel plate fixture with 25 mm diameter at gap of 1 mm. The strain amplitude was varied logarithmically from 0.001 to 100% at constant frequency of 1 Hz (or 10 rad/s) and 25° C. temperature. The Yield value was determined to be 933.2 Pa and Flow point to be 1327 Pa.

### Example 5

Preparation of lyophilized mixture containing
liraglutide with arginine

| | | Quantity |
|---|---|---|
| S. No. | Ingredients | % w/w |
| 1 | Liraglutide (acetate) equivalent to liraglutide | 2.5 |
| 2 | Arginine | q.s to pH 7. [symbol] |
| 3 | Water for Injection | 100 |

[symbol]

indicates data missing or illegible when filed

Water for injection was taken in a vial. Required first part quantity of arginine was dissolved in water for injection under gentle stiffing. Liraglutide acetate was added to above solution with stiffing. Remaining arginine was added while stirring until a pH of 7.0 was obtained and the solution was clear. The aqueous solution was then filtered with 0.2 μm membrane filter, filled into vials and lyophilized. The assay for liraglutide in the lyophilized mixture was 99.9%.

### Example 6

### Preparation of Aqueous Solution at Second Concentration from Lyophilized Mixture Containing Liraglutide with Arginine

Three different solutions at second concentration were prepared as follows:

(a) Aqueous solution at of liraglutide
at second concentration (28.6% w/w)

| | Ingredients | |
|---|---|---|
| Sr. No. | Aqueous solution at second concentration | Quantity (mg) |
| 1 | Lyophilized mixture of Example 5 | 48* |
| 2 | Sterile Water for injection | 120 |

*equivalent to liraglutide base

Lyophilized mixture of Example 5 was dissolved in sterile water for injection resulting in aqueous solution with second concentration (28.6% w/w) having a pH of 7. This was kept at 20-25° C.

(b) Aqueous solution at of liraglutide
at second concentration (39.4% w/w)

| | Ingredients | |
|---|---|---|
| Sr. No. | Aqueous solution at second concentration | Quantity (mg) |
| 1 | Lyophilized mixture of Example 5 | 26* |
| 2 | Sterile Water for injection | 40 |

*equivalent to liraglutide base

Lyophilized mixture of Example 5 was dissolved in sterile water for injection resulting in aqueous solution with second concentration (39.4% w/w) having a pH of 7. This was kept at 20-25° C.

(c) Aqueous solution of liraglutide
at second concentration (44.4% w/w)

| | Ingredients | |
|---|---|---|
| Sr. No. | Aqueous solution at second concentration | Quantity(mg) |
| 1 | Lyophilized mixture of Example 5 | 32* |
| 2 | Sterile Water for injection | 40 |

*equivalent to liraglutide base

Lyophilized mixture of Example 5 was dissolved in sterile water for injection resulting in aqueous solution with second concentration (44.4% w/w) having a pH of 7. This was kept at 20-25° C.

(d) Aqueous solution at of liraglutide
at second concentration (48.7% w/w)

| | Ingredients | |
|---|---|---|
| Sr. No. | Aqueous solution at second concentration | Quantity(mg) |
| 1 | Lyophilized mixture of Example 5 | 38* |
| 2 | Sterile Water for injection | 40 |

*equivalent to liraglutide base

Lyophilized mixture of Example 5 was dissolved in sterile water for injection resulting in aqueous solution with second concentration (48.7% w/w) having a pH of 7. This was kept at 20-25° C.

### Example 7

Preparation Gel composition using aqueous solution
of second concentration of Example 6

| | | Quantity |
|---|---|---|
| S. No | Ingredients | % w/w |
| 1 | Liraglutide | 15 |
| | Non-aqueous solution of amphipaths | |
| 1 | Soy Phosphatidylcholine (Lipoid S 100) | 24.6 |
| 2 | Glycerol Oleates in ratio: - | 24.6 |
| | GMO:GDO:GTO = 44:42:9 | |
| | (IMWITOR ® 948) | |
| 3 | Ethanol Absolute 99.9% | 5.8 |
| 4 | Propylene Glycol USP | 2.9 |
| | Total preparation | 100 |

Non-Aqueous Solution of Amphipaths—

Soy Phosphatidylcholine (Lipoid S 100), Glycerol Oleate (IMWITOR® 948, mixture of mono, di and tri oleate and free fatty acid) were dissolved in Propylene Glycol and Ethanol at 60-70° C. with stiffing for 15-20 min and filtered with 0.2 μm membrane filter.

Gel Composition—

Non-aqueous solution of amphipaths was added to 42.1% w/w of aqueous solution at second concentration of Example 6(b) (comprising liraglutide at a concentration of 39.4% w/w) and mixed using stirrer, to form gel using stiffing at 50-70° C. The lipid gel is yellowish viscous of semisolid consistency. The yield value and flow point were measured by Anton Paar MCR 302 rheometer using parallel plate fixture with 25 mm diameter at gap of 1 mm. The strain amplitude was varied logarithmically from 0.001 to 100% at constant frequency of 1 Hz (or 10 rad/s) and 25° C. temperature. The Yield value was determined to be 1805 Pa and Flow point to be 2396 Pa.

### Example 8

Preparation Gel composition using aqueous solution
of second concentration of Example 6

| | | Quantity |
|---|---|---|
| S. No | Ingredients | % w/w |
| 1 | Liraglutide | 20 |
| | Non-aqueous solution of amphipaths | |
| 1 | Soy Phosphatidylcholine (Lipoid S 100) | 22.7 |
| 2 | Glycerol Oleates in ratio: - GMO:GDO:GTO = | 21.2 |
| | 44:42:9 (IMWITOR ® 948) | |
| 3 | Ethanol Absolute 99.9% | 1.6 |
| 4 | Propylene Glycol USP | 5.3 |
| | Total preparation | 100 |

Non-aqueous solution of amphipaths - Soy Phosphatidylcholine (Lipoid S 100), Glycerol Oleate (IMWITOR ® 948, mixture of mono, di and tri oleate and free fatty acid) were dissolved in Propylene Glycol and Ethanol at 60-70° C. with stirring for 15-20 min and filtered with 0.2 μm membrane filter.
Gel composition- Non-aqueous solution of amphipaths was added to 46.5% w/w of aqueous solution at second concentration of Example 6(c) (comprising liraglutide at a concentration of 44.4% w/w) and mixed using stirrer, to form gel using stirring at 50-70° C. The lipid gel is yellowish viscous of semisolid consistency. The yield value and flow point were measured by Anton Paar MCR 302 rheometer using parallel plate fixture with 25 mm diameter at gap of 1 mm. The strain amplitude was varied logarithmically from 0.001 to 100% at constant frequency of 1 Hz (or 10 rad/s) and 25° C. temperature. The Yield value was determined to be 1902 Pa and Flow point to be 2448 Pa.

### Example 9

Preparation Gel composition using aqueous solution
of second concentration of Example 6

| | | Quantity |
|---|---|---|
| S. No | Ingredients | % w/w |
| 1 | Liraglutide | 25 |
| | Non-aqueous solution of amphipaths | |
| 1 | Soy Phosphatidylcholine (Lipoid S 100) | 18.45 |
| 2 | Glycerol Oleates in ratio: - GMO:GDO:GTO = | 18.45 |
| | 44:42:9 (IMWITOR ® 948) | |
| 3 | Ethanol Absolute 99.9% | 3.95 |
| 4 | Propylene Glycol USP | 1.98 |
| | Total preparation | 100 |

Non-aqueous solution of amphipaths - Soy Phosphatidylcholine (Lipoid S 100), Glycerol Oleate (IMWITOR ® 948, mixture of mono, di and tri oleate and free fatty acid) were dissolved in Propylene Glycol and Ethanol at 60-70° C. with stirring for 15-20 min and filtered with 0.2 μm membrane filter.
Gel composition- Non-aqueous solution of amphipaths was added to 57.2% w/w of aqueous solution at second concentration of Example 6(d) (comprising liraglutide at a concentration of 48.7% w/w) and mixed using stirrer, to form gel using stirring at 50-70° C. The lipid gel is yellowish viscous of semisolid consistency

### Example 10

| | | Quantity |
|---|---|---|
| Sr. No. | Ingredients | (% w/w) |
| 1 | Liraglutide sodium | 25.00 |
| 2 | Water for injection | 21.81 |
| 3 | Soy Phosphatidylcholine (Lipoid S 100) | 22.61 |
| 4 | Glycerol Oleates mixture (IMWITOR ® 948) | 21.06 |
| 5 | Ethanol Absolute 99.9% | 5.32 |
| 6 | Propylene Glycol USP, | 2.66 |
| 7 | Glyceryl trioleate (GTO) | 1.54 |
| | Total gel phase | 100 |

An Aqueous phase was prepared by dissolving liraglutide (liraglutide Na equivalent to Liraglutide) in water for injection. The aqueous phase is kept at 20-25° C. Separately, a lipid phase was prepared by mixing Soy Phosphatidylcholine (Lipoid S 100), Glycerol Oleates mixture of mono, di and tri oleate and free fatty acid—IMWITOR® 948), Glyceryl trioleate (GTO), Propylene Glycol and Ethanol at 60-70° C. under stiffing for 20-40 min and filtered with 0.2 μm membrane filter. The lipid phase was then added to the aqueous phase and mixed using stiffing at 50-70° C. temperature to form lipid gel.

### Example 11

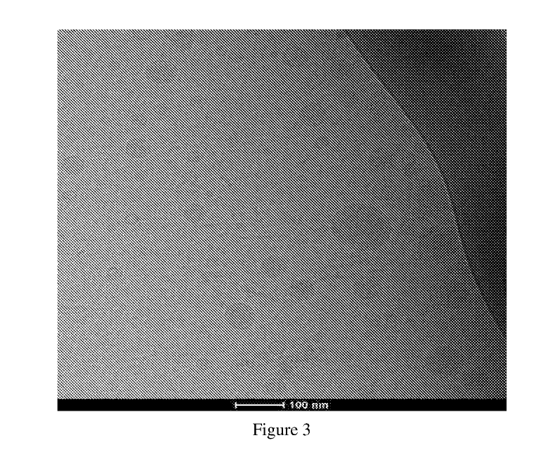

In order to simulate the formation of a depot when the parenteral composition is injected, the morphology of Gel of Example 4 when dispersed in WFI was examined using transmission electron microscopic (TEM) technique. Lipid Gel of Example 4 was dispersed in WFI (pH between 6.0 to 7.0) in 1.0 mg/ml concentration at 40-60° C. temperature under stiffing for 5-10 min and sonication for 5-10 min to form unique phase. FEI's Tecnai™ Spirit cryo-transmission electron microscope was used for morphology study. The sample of gel dispersion was dropped on standard carbon coated copper grid (mesh) and air dried. TEM images were viewed under transmission electron microscope operating at an accelerated voltage of 120 kV and analyzed. The gel of Example 4 shows cubic structure forms when gel is dispersed into the water for injection. The dense cubic phase also was observed together with few lamellar particles (Vesicles) (See FIG. 3). This represents the mechanism by which the gel would disintegrate upon injection at subcutaneous site.

### Example 12

Preclinical efficacy study was performed on the db/db mice model of type 2 diabetes. The animals were acclimatized for 5 days. On day 0, each animal was weighed. The baseline value was determined by collecting approximately 100 μL of blood from Preclinical efficacy study was performed on the db/db mice model of type II diabetes. All the animals were acclimatized for 5 day. On day 0, each animal was weighed and approximately 10 μL of blood was collected from retro-orbital plexus and blood glucose concentration was measured with glucose strips using Blood Glucose Meter (Blood Glucose Meter, One Touch™ Ultra™; LIFESCAN, Johnson & Johnson) This was considered as baseline value (0 hour). The animals were randomized into treatment groups containing 4 male and 4 female animals each. The single calculated dose of gel of Example 4 (10 mg/kg) was injected subcutaneously in the neck region of the animals. The blood was collected and blood glucose concentration was measured at 1, 4, 8, 12, 24, 48, 96, 144 and 168 hour post injection. The data was analysed using PRISM (Graph Pad version 5.04 Dec. 10, 2011) by Student's paired t-test as compared to baseline. It was observed that gel composition (Gel 10%) of Example 4 gave significantly better glucose lowering effects for 7 days compared to Comparative Example 1 (comparative) as demonstrated in FIGS. 4(a) and 4(b) resp.

### Example 13

Multiple dose efficacy study—Preclinical efficacy study was performed as per the procedure described in Example 10. Gel composition (Gel 10%) of Example 4 was injected weekly on day 0, 7, 14, and 21 at 10 mg/kg liraglutide (5 times human eq. dose). The blood was collected at 0 d (1 h, 4 h, 8 h, 12 h 1 d, 2 d, 4 d, 6 d, 7 d predose), 7 d (1 h, 4 h, 8 h, 12 h, 8 d, 9 d, 11 d, 13, 14 d predose), 14 d (1 h, 4 h, 8 h, 12 h, 15 d, 16 d, 18 d, 20 d, 21 d pre-dose) and 21 d (1 h, 4 h, 8 h, 12 h, 22 d, 23 d, 25 d, 27 d, 28 d) intervals post injection and blood glucose level was measured. Gel composition (Gel 10%) of Example 4 showed significant reduction in % blood glucose levels up to 4 weeks as shown in FIG. 5 as compared to placebo.

### Example 14

Multiple dose preclinical efficacy study for Gel composition (Gel 10%) of Example 4 was performed on the Diet Induced rat model (for insulin-resistant T2D) of type 2 diabetes. Preclinical efficacy study was performed as per the procedure described in Example 10. Gel composition of Example 4 was compared with Victoza®. Victoza® was injected daily for 28 days at a human eq. dose of 0.2 mg/kg (1.8 mg is approved dose of Victoza). Gel composition of Example 4 was injected weekly on day 0, 7, 14, and 21 at 10 mg/kg liraglutide (5 times human eq. dose). The blood was collected on 0 d (1, 4, 8, 12 h), 1 d, 2 d, 4 d, 6 d, 7 d (1, 4, 8, 12 h), 8 d, 9 d, 11 d, 13 d, 14 d (1, 4, 8, 12 h), 15 d, 16 d, 18 d, 21 d (1, 4, 8, 12 h), 22 d, 23 d, 25 d, 27 d and 28 d intervals post injection and blood glucose levels, % HbA1C was measured on day 0, 14, and 28 d. % HbA1C was measured using kits (BioSystem, Spain).

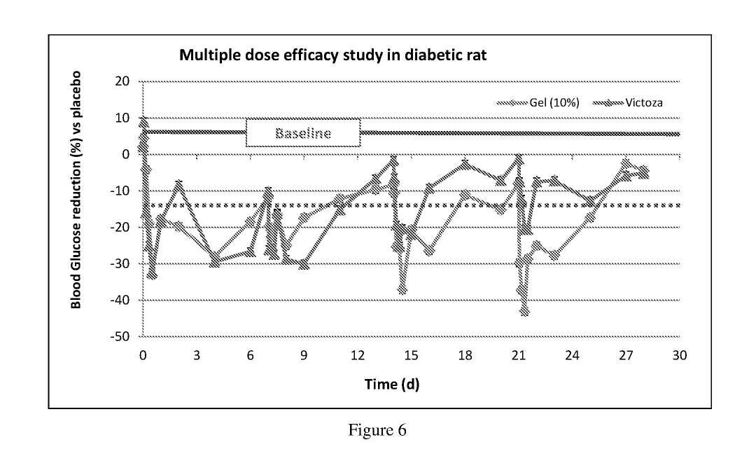

The results are shown in FIG. 6, where gel composition (Gel 10%) of Example 4 and marketed liraglutide solution (Victoza®) showed significant reduction in % blood glucose levels up to 4-weeks; the optimum reduction in blood glucose levels were −43.07% and −32.35% respectively. The HbA1C results are shown in FIG. 7. HbA1C reduction as 1.86%, and 1.85% respectively for gel composition (Gel 10%) of Example 4 and Victoza® compared to placebo.

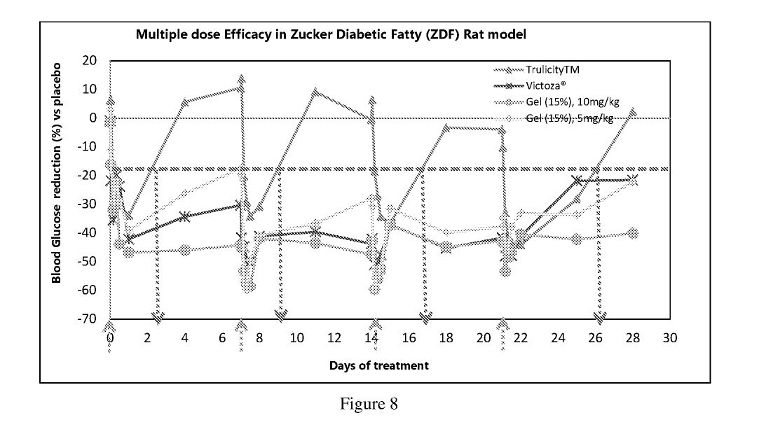

Similarly, multiple dose preclinical efficacy study for Gel composition (Gel 15%) of Example 7 was performed on the Zucker Diabetic Fatty (ZDF) T2D rat model as per the procedure described above. The result are shown in FIG. 8 where gel composition (Gel 15%) in two different doses (5 & 10 mg/kg), solution (Victoza® daily injection) and solution (Trulicity™ weekly injection) showed significant reduction in % blood glucose levels, Lipid gel & Victoza® showed the significant reduction in % blood glucose levels up to 4-weeks; while Trulicity™ showed the significant reduction up to 2.5-3 days after each weekly SC administration only. The HbA1C results are shown in FIG. 9. % HbA1C reduction as 2.37, 2.51, 2.36 and 1.03 respectively for gel composition in two different doses (5 & 10 mg/kg), solution (Victoza® daily injection) and solution (Trulicity™ weekly injection) compared to placebo.

### Example 15

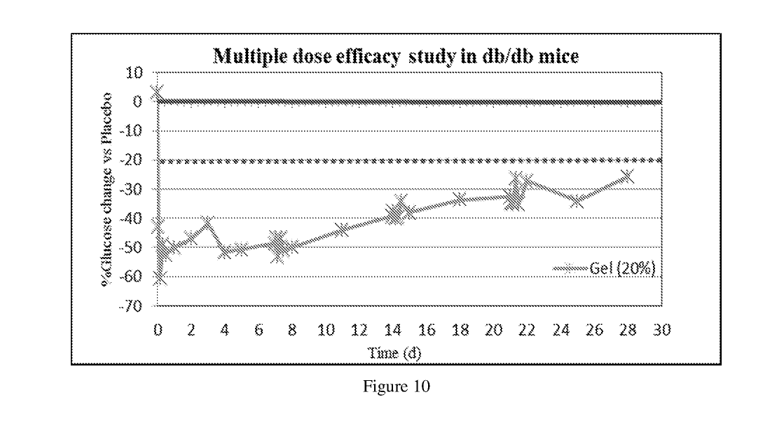

Preclinical efficacy study was performed as per the procedure described above in Example 12. The blood was collected on 0 d (0, 1, 4, 8, 12 h), 1 d, 2 d, 3 d, 4 d, 5 d, 7 d (0, 1, 4, 8, 12 h), 8 d, 11 d, 14 d (0, 1, 4, 8, 12 h), 15 d, 18 d, 21 d (0, 1, 4, 8, 12 h), 22 d, 25 d and 28 d intervals post injection and blood glucose levels was measured. Gel composition (Gel 20%) of Example 8 was injected weekly on day 0, 7, 14, and 21 at 10 mg/kg liraglutide (4 times human eq. dose) and blood glucose levels was measured. Gel composition (Gel 20%) showed significant reduction in % blood glucose levels up to 4 weeks as shown in FIG. 10.

We claim:

1. A viscoelastic gel comprising a therapeutically effective amount of liraglutide wherein the gel does not comprise a block or a graft copolymer or mixtures thereof and wherein the gel is characterized by a yield value from 200 Pa to 3000 Pa and a flow Point from 300 Pa to 3500 Pa.

2. The viscoelastic gel as in claim 1, wherein liraglutide is present at a concentration from about 5% to 30% by weight of the gel.

3. The viscoelastic gel as in claim 2, comprising liraglutide at a concentration from about 10% to 25% by weight of the gel, at least one amphipath and an aqueous vehicle.

4. The viscoelastic gel as in claim 3, wherein the amphipath is present at a concentration from 40% to 60% by weight of the gel and the aqueous vehicle is present at a concentration from 20% to 40% by weight of the gel.

5. The viscoelastic gel as in claim 3, wherein the amphipath is selected from glyceryl monooleate, glyceryl dioleate, glyceryl trioleate, polyglyceryl-3-dioleate, phosphatidylcholine, and mixtures thereof.

6. The viscoelastic gel as in claim 5, wherein the amphipath is a mixture of glyceryl monooleate, glyceryl dioleate, glyceryl trioleate and phosphatidylcholine.

7. The viscoelastic gel as in claim 3, wherein the aqueous vehicle is a mixture of water and a water miscible solvent.

8. The viscoelastic gel as in claim 3 wherein the viscoelastic gel further comprises parenterally acceptable amine base

9. The viscoelastic gel as in claim 8, the parenterally acceptable amine base is selected from tromethamine, arginine, histidine, lysine, guanidine, epolamine, glucosamine and meglumine

10. A method of controlling blood glucose levels in a subject in need of control of blood glucose levels, the method comprising subcutaneously administering once weekly or bi-weekly to the subject a viscoelastic gel comprising a therapeutically effective amount of liraglutide, the gel does not comprise a block or a graft copolymer or mixtures thereof and the gel is characterized by a yield value from 200 Pa to 3000 Pa and a flow Point from 300 Pa to 3500 Pa.

11. The method as in claim 10, wherein the viscoelastic gel comprises liraglutide at a concentration from about 5% to 30% by weight of the gel.

12. The method as in claim 11, wherein the viscoelastic gel comprises liraglutide at a concentration from about 10% to 25% by weight of the gel, at least one amphipath, and an aqueous vehicle.

13. The method as in claim 12, wherein the amphipath is present at a concentration from 40% to 60% by weight of the gel and the aqueous vehicle is present at a concentration from 20% to 40% by weight of the gel.

14. The method as in claim 12, wherein the amphipath is selected from glyceryl monooleate, glyceryl dioleate, glyceryl trioleate, polyglyceryl-3-dioleate, phosphatidylcholine, and mixtures thereof.

15. The method as in claim 14, wherein the amphipath is a mixture of glyceryl monooleate, glyceryl dioleate, glyceryl trioleate and phosphatidylcholine.

16. The method as in claim 12, wherein the aqueous vehicle is a mixture of water and a water miscible solvent.

17. The method as in claim 12 wherein the viscoelastic gel further comprises parenterally A acceptable amine base

18. The method as in claim 17, the parenterally acceptable amine base is selected from tromethamine, arginine, histidine, lysine, guanidine, epolamine, glucosamine and meglumine
